(19) United States
(12) Patent Application Publication
(10) Pub. No.: US 2021/0361150 A1
(43) Pub. Date: Nov. 25, 2021

(54) MEDICAL INSTRUMENT

(71) Applicant: OLYMPUS CORPORATION, Tokyo (JP)

(72) Inventor: Takashi NAKAMURA, Tokyo (JP)

(73) Assignee: OLYMPUS CORPORATION, Tokyo (JP)

(21) Appl. No.: 17/392,494

(22) Filed: Aug. 3, 2021

Related U.S. Application Data
(63) Continuation of application No. PCT/JP2019/004726, filed on Feb. 8, 2019.

(51) Int. Cl. A61B 1/018; A61B 1/00

(52) U.S. Cl. CPC A61B 1/018; A61B 1/00025; A61B 1/00133; A61B 1/00135; A61B 1/00087

(57) ABSTRACT

A medical instrument includes a main body, a first tubular member configured to transmit power by rotating or periodically deforming, and a second tubular member configured to be attached to or detached from the first tubular member, in which the second tubular member includes an external gear member disposed on an outside of the first tubular member in a radial direction, and configured to have a plurality of external teeth arranged in a circumferential direction on an outer circumferential surface and to swing or deform according to power of the first tubular member, and an internal gear member disposed on an outside of the external gear member in the radial direction and configured to have a plurality of internal teeth arranged in the circumferential direction on an inner circumferential surface, and the number of internal teeth is greater than the number of external teeth.

This application is a continuation application based on a PCT International Application No. PCT/JP2019/004726, filed on Feb. 8, 2019. The content of the PCT International Application is incorporated herein by reference.

### BACKGROUND OF THE INVENTION

### Field of the Invention

The present invention relates to a medical instrument provided with a medical rotation mechanism provided in the medical instrument.

### Description of Related Art

There is known a medical rotation mechanism that assists a treatment of inserting a medical instrument such as an endoscope device having an insertion portion provided with an imaging unit for observing an image in a lumen at a distal end thereof into the lumen.

United States Patent Application, Publication No. 2012/0029281 describes an endoscope device provided with a medical rotation mechanism that rotates about a longitudinal axis in an insertion portion.

In addition, Japanese Patent No. 5458224 describes an in-vivo introduction device provided with a rotation mechanism that rotates a spiral fin in an insertion portion. The in-vivo introduction device rotates the rotation mechanism connected to a shaft inside the insertion portion to rotate the spiral fin provided on the outside of the insertion portion. The in-vivo introduction device assists the insertion of the insertion portion into the lumen by rotating the spiral fin.

### SUMMARY OF THE INVENTION

According to a first aspect of the present invention, there is provided a medical instrument including a main body, a first tubular member provided on the main body and configured to transmit power by rotating or periodically deforming, and a second tubular member configured to be attached to or detached from the first tubular member and which is a tubular member, in which the second tubular member includes an external gear member disposed on an outside of the first tubular member in a radial direction, and configured to have a plurality of external teeth arranged in a circumferential direction on an outer circumferential surface and to swing or deform according to power of the first tubular member, and an internal gear member disposed on an outside of the external gear member in the radial direction and configured to have a plurality of internal teeth arranged in the circumferential direction on an inner circumferential surface, and the number of internal teeth is greater than the number of external teeth.

### BRIEF DESCRIPTION OF THE DRAWINGS

FIG. 1 is a diagram showing a conceptual external configuration of an endoscope device according to a first embodiment of the present invention.

FIG. 2 is a perspective view of a medical rotation mechanism of the endoscope device.

FIG. 3 is a perspective view of the medical rotation mechanism of the endoscope device.

FIG. 4 is a perspective view of the medical rotation mechanism when a rotary member (second tubular member) is attached.

FIG. 5 is a cross-sectional view of an A-A cross section of the medical rotation mechanism.

FIG. 6 is a cross-sectional view of the medical rotation mechanism that transmits rotational power to the rotary member.

FIG. 7 is a cross-sectional view of the rotary member.

FIG. 8 is a cross-sectional view of the medical rotation mechanism that transmits the rotational power to the rotary member.

FIG. 9 is a cross-sectional view of the medical rotation mechanism that transmits the rotational power to the rotary member.

FIG. 10 is a cross-sectional view of the medical rotation mechanism that transmits the rotational power to the rotary member.

FIG. 11 is a perspective view showing a modification example of a wave generator of the medical rotation mechanism.

FIG. 12 is a cross-sectional view of a medical rotation mechanism of an endoscope device according to a second embodiment of the present invention.

FIG. 13 is a diagram showing an external configuration of a treatment tool according to a third embodiment of the present invention.

FIG. 14 is a diagram showing an external configuration of a treatment tool according to a fourth embodiment of the present invention.

### DETAILED DESCRIPTION OF THE INVENTION

### First Embodiment

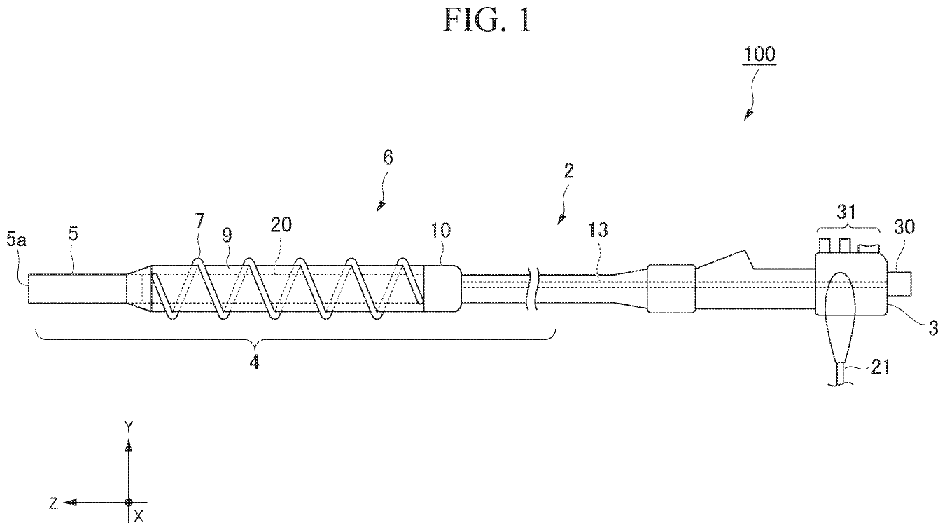

The first embodiment of the present invention will be described with reference to FIGS. 1 to 10.

FIG. 1 is a diagram showing a conceptual external configuration of an endoscope device 100 according to the present embodiment.

As shown in FIG. 1, the endoscope device (medical instrument) 100 is provided with an insertion portion 2 inserted into a lumen of a living body and an operation portion 3 provided on a base end side of the insertion portion 2.

As shown in FIG. 1, the insertion portion 2 is provided with a long insertion portion main body (main body) 4 extending along the longitudinal axis direction of the insertion portion 2, a curved portion 5 provided on a distal end side of the insertion portion main body 4, an in-vivo insertion mechanism 6, and a medical rotation mechanism 10.

The curved portion 5 is a long member that curves according to bending of the lumen. An imaging unit (not shown) is provided at a distal end portion 5a of the curved portion 5. The insertion portion 2 is provided with a channel 20 which is a passage (internal cavity) extending from the distal end portion 5a to the entire length of the insertion portion 2. A treatment tool such as a high-frequency knife or grasping forceps is inserted into the channel 20.

The in-vivo insertion mechanism 6 is a tubular member that fits on the outer circumference of the insertion portion main body 4 or the curved portion 5 with a gap, and is attached to or detached from the medical rotation mechanism 10. The in-vivo insertion mechanism 6 includes a fin 7 that functions as a propulsion portion and a retreat portion, and a spiral tube (introduction propulsion unit) 9 that rotates about the longitudinal axis and functions as introduction propulsion.

The fin 7 is spirally wound around the outer circumference of the spiral tube 9. By rotating the spiral tube 9, the in-vivo insertion mechanism 6 moves forward and rearward in the lumen.

The spiral tube 9 has a material (for example, a rubber material or a resin material) or a structure having flexibility to follow the curvature of the curved portion 5. A distal end side of the spiral tube 9 is formed in a tapered shape and can be easily inserted into the lumen.

The in-vivo insertion mechanism 6 is a disposable product that is attached to or detached from the medical rotation mechanism 10, and can be replaced for each treatment to prevent infection.

The medical rotation mechanism 10 is attached to the insertion portion main body 4 and rotates the spiral tube 9 about the longitudinal axis of the insertion portion 2 to assist the introduction of the insertion portion 2 into the lumen. The medical rotation mechanism 10 can rotate the spiral tube 9 in both directions (CW and CCW).

A first end of the shaft 13 that inserts the inside of the insertion portion 2 is connected to the medical rotation mechanism 10, and a second end of the shaft 13 is connected to a motor (not shown) provided in the operation portion 3. The motor rotates the shaft 13 about the longitudinal axis to rotate a part of the medical rotation mechanism 10.

The operation portion 3 is provided with a knob 30 and a switch 31 for performing various operations including the bending operation of the curved portion 5 and the rotation of the medical rotation mechanism 10.

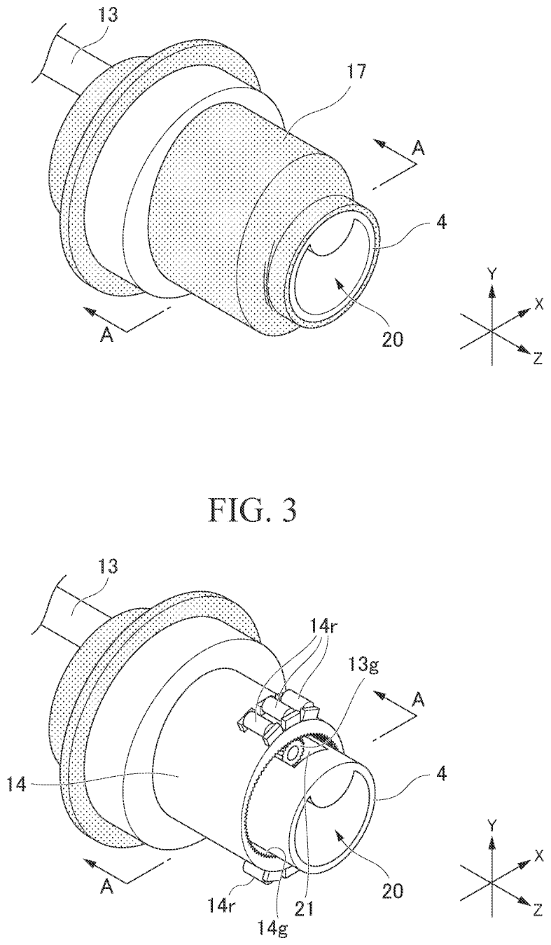

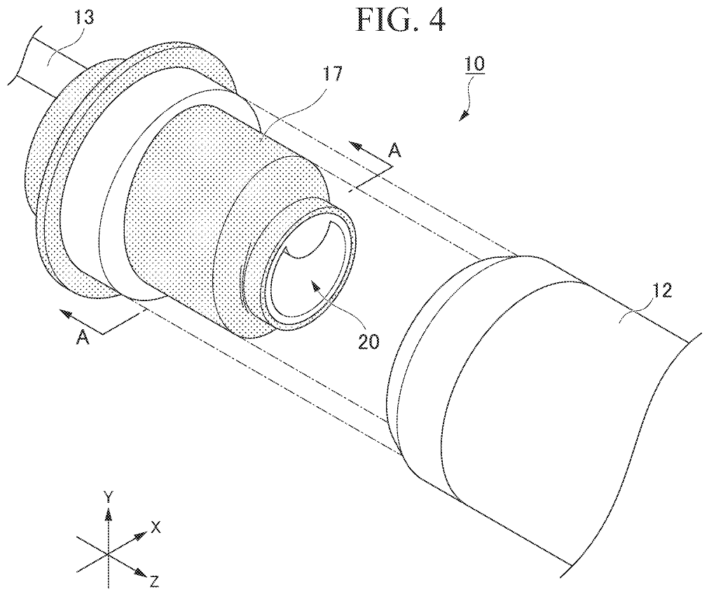

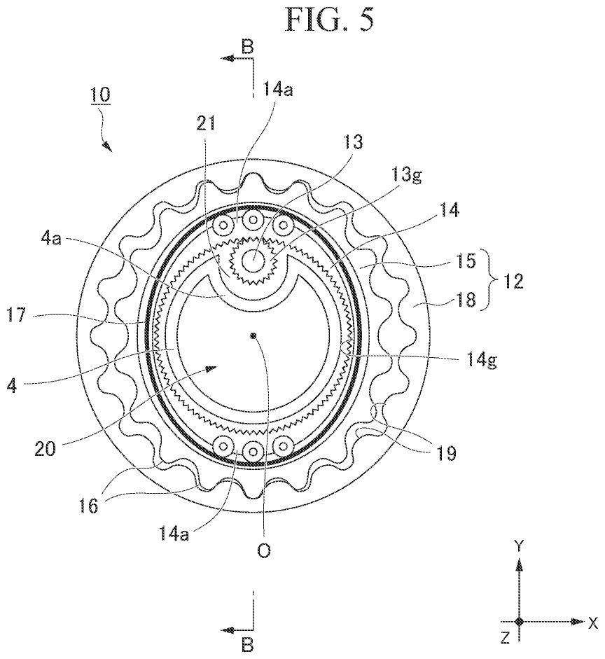

FIG. 2 is a perspective view of the medical rotation mechanism 10 from which the rotary member 12 is removed. FIG. 3 is a perspective view of the medical rotation mechanism 10 from which the covering member 17 and the rotary member 12 are removed. FIG. 4 is a perspective view of the medical rotation mechanism 10 when the rotary member 12 is attached. FIG. 5 is a cross-sectional view of an A-A cross section (cross section perpendicular to the longitudinal direction of the insertion portion 2) of the medical rotation mechanism 10 shown in FIG. 2. In the following description, the A-A cross section is also referred to as an XY plane, and the longitudinal axis direction of the insertion portion 2 is also referred to as a Z-axis direction.

As shown in FIG. 5, the medical rotation mechanism 10 includes a drive gear 13g connected to the shaft 13, a wave generator (first tubular member) 14 inscribed and intermeshed with the drive gear 13g, a covering member 17 covering the wave generator 14, and a rotary member (second tubular member) 12 disposed on the outside of the wave generator 14 in the radial direction.

The shaft 13 is disposed inside a gear cylinder 4a that forms a cavity 21 separated from the channel 20 of the insertion portion 2. The cavity 21 forms a path extending from the base end of the insertion portion 2 to the medical rotation mechanism 10. In addition, as shown in FIGS. 3 and 5, the cavity 21 communicates with the internal space of the wave generator 14 at least in the A-A cross section. The drive gear 13g is connected to the end portion of the shaft 13.

As shown in FIG. 5, the wave generator (first tubular member) 14 is a cylindrical member having transmission gears 14g arranged in the circumferential direction on the inner circumferential surface. The transmission gear 14g is a gear that inscribes and meshes with the drive gear 13g. The wave generator 14 is rotatably supported about the longitudinal axis by the insertion portion main body 4. The rotation axis of the wave generator 14 is hereinafter referred to as “rotation axis O”. The wave generator 14 rotates about the rotation axis O in accordance with the rotation of the drive gear 13g in which the transmission gear 14g is inscribed and intermeshed.

As shown in FIG. 5, the wave generator 14 has an elliptical shape on the XY plane, and has two cam portions 14a having a length in the radial direction longer than that of the other portions in the circumferential direction in a part in the circumferential direction. The two cam portions 14a are elliptical long-shaft portions, and are disposed at positions facing each other with the central axis O interposed therebetween.

When the wave generator 14 rotates about the rotation axis O, the cam portion 14a moves in the circumferential direction. The wave generator 14 transmits rotational power about the rotation axis O of the wave generator 14 to the rotary member 12 disposed outside the wave generator 14.

A roller 14r is provided at the distal end of the cam portion 14a. The roller 14r is rotatably supported about the Z-axis direction. A plurality of rollers 14r are arranged in the circumferential direction, and three rollers are disposed to each side of the distal ends of two cam portions 14a facing each other with the central axis O interposed therebetween. The wave generator 14 brings the cam portion 14a into contact with the rotary member 12 disposed outside the wave generator 14 via the roller 14r, and transmits rotational power about the central axis O of the wave generator 14 to the rotary member 12.

As shown in FIGS. 2 and 5, the covering member 17 is an elastic member such as rubber disposed between the wave generator 14 and the rotary member 12, and covers the wave generator 14 to isolate the wave generator 14 from the outside world, and makes the inside of the wave generator 14 watertight.

The rotary member 12 is a cylindrical member that can be attached to or detached from the wave generator 14, and includes an external gear member 15 and an internal gear member 18 disposed on the outside of the external gear member 15 in the radial direction.

As shown in FIG. 5, the external gear member 15 is a thin-walled tubular member disposed on the outside of the wave generator 14 in the radial direction, and is made of an elastic member such as metal or rubber. The external gear member 15 is non-rotatably supported with respect to the insertion portion main body 4. The external gear member 15 is deformed according to the power of the wave generator 14.

The external gear member 15 includes a plurality of external teeth 16 arranged in the circumferential direction on an outer circumferential surface. On the outer circumferential surface of the external gear member 15, the external teeth 16 are evenly disposed in the circumferential direction, and the outer circumferential surface including the external teeth 16 forms a cycloid curve or a cycloid parallel curve along the circumferential direction. The number of external teeth 16 is 18. In the present embodiment, although the outer circumferential surface of the external gear member 15 forms a cycloid curve or a cycloid parallel curve, an involute curve may be formed.

As shown in FIG. 5, the internal gear member 18 is a tubular member disposed on the outside of the external gear member 15 in the radial direction, and is made of a highly rigid metal or the like. The internal gear member 18 is rotatably supported with respect to the insertion portion main body 4 about the rotation axis O. The internal gear member 18 is connected to the spiral tube 9, and when the internal gear member 18 rotates about the rotation axis O, the spiral tube 9 also rotates about the rotation axis O. The internal gear member 18 and the spiral tube 9 may be integrally formed.

The internal gear member 18 includes a plurality of internal teeth 19 arranged in the circumferential direction on the inner circumferential surface. As shown in FIG. 5, on the inner circumferential surface of the internal gear member 18, the internal teeth 19 are evenly disposed in the circumferential direction, and the inner circumferential surface including the internal teeth 19 forms a cycloid curve or a cycloid parallel curve along the circumferential direction. As shown in FIG. 5, the number of internal teeth 19 included in the internal gear member 18 is 20. On the other hand, the number of external teeth 16 included in the external gear member 15 is 18. That is, the number of internal teeth 19 is two more than the number of external teeth 16. The external gear member 15 and the internal gear member 18 function as “wave gears”.

The number of external teeth 16 included in the external gear member 15 and the number of internal teeth 19 included in the internal gear member 18 are not limited thereto. The number of internal teeth 19 may be more than the number of external teeth 16 by two or more. For example, the number of external teeth 16 may be (number of internal teeth−4)=(20−4)=16.

As shown in FIG. 5, the external teeth 16 on the outer circumferential side of the external gear member 15 with which the cam portion 14a of the wave generator 14 is in contact are inscribed and intermeshed with the internal teeth 19. The wave generator 14 causes the external teeth 16 and the internal teeth 19 to be inscribed and intermeshed with each other, a portion where the external teeth 16 and the internal teeth 19 are inscribed and intermeshed with each other (hereinafter, also referred to as “inscribed meshing portion E”) to be moved in the circumferential direction, and the rotational power about the rotation axis O to transmit to the rotary member 12. As a result, the internal gear member 18 rotates about the rotation axis O. In the present embodiment, the rotary member 12 includes two inscribed meshing portions E, and the two inscribed meshing portions E are disposed at positions facing each other with the central axis O interposed therebetween. The inner circumference of the external gear member 15 is extruded by the wave generator 14 to form an elliptical shape.

As shown in FIG. 4, the rotary member 12 is attached to or detached from the endoscope device 100 on the outside of the covering member 17. The rotary member 12 is attached to the endoscope device 100 by fitting the rotary member 12 on the outer circumference of the covering member 17 with a gap. The rotary member 12 is a disposable product that is attached to or detached from the endoscope device 100, and can be replaced for each treatment to prevent infection.

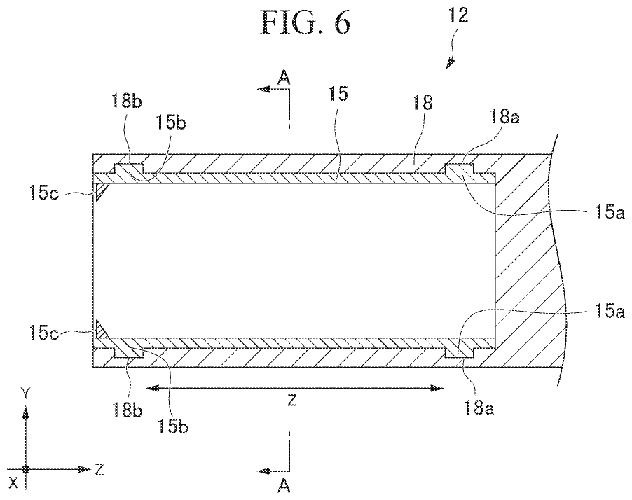

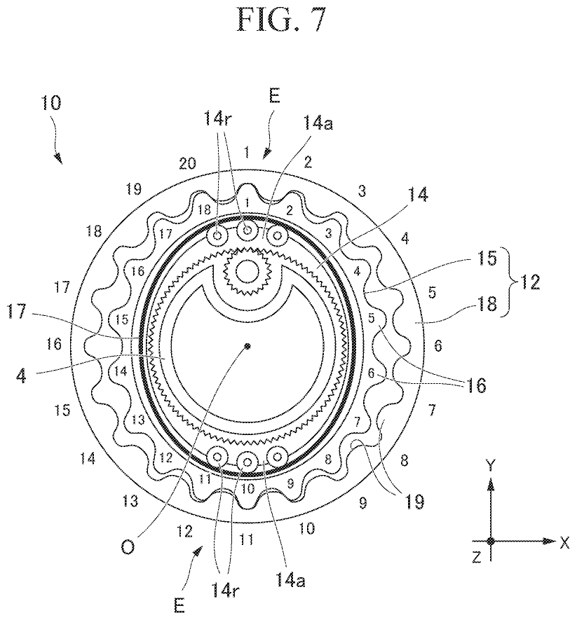

FIG. 6 is a cross-sectional view of the rotary member 12 in a B-B cross section (cross section horizontal to the longitudinal direction of the insertion portion 2) shown in FIG. 5. The rotary member 12 shown in FIG. 6 is removed from the wave generator 14.

The internal gear member 18 includes a recessed portion 18a on a distal end side and a recessed portion 18b on a base end side on the inner circumferential surface. The recessed portion 18a and the recessed portion 18b are recessed portions formed in annular shapes on the inner circumferential surface of the internal gear member 18.

The external gear member 15 includes a projection portion 15a on a distal end side and a projection portion 15b on a base end side on the outer circumferential surface. The projection portion 15a and the projection portion 15b are projection portions formed in annular shapes on the outer circumferential surface of the external gear member 15.

When the rotary member 12 is attached to the wave generator 14, the recessed portion 18a and the projection portion 15a are engaged with each other, and the recessed portion 18b and the projection portion 15b are engaged with each other. As a result, the relative positions of the internal gear member 18 and the external gear member 15 can be preferably maintained.

The external gear member 15 includes a projection portion 15c, which is a projection portion formed in an annular shape on the base end side on the inner circumferential surface. The projection portion 15c functions as a retainer for preventing the rotary member 12 from coming off from the wave generator 14 when the rotary member 12 is attached to the wave generator 14.

The internal teeth 19 are provided in the region Z between the recessed portion 18a and the recessed portion 18b in the Z-axis direction (longitudinal axis direction of the insertion portion 2).

The external teeth 16 are provided in the region Z between the projection portion 15a and the projection portion 15b in the Z-axis direction (longitudinal axis direction of the insertion portion 2).

The internal teeth 19 and the external teeth 16 mesh with each other in the region Z between a distal end side engaging portion where the recessed portion 18a and the projection portion 15a engage with each other and a base end side engaging portion where the recessed portion 18b and the projection portion 15b engage with each other. Therefore, the recessed portion 18a and the recessed portion 18b, and the projection portion 15a and the projection portion 15b are surely intermeshed with the internal teeth 19 and the external teeth 16, and the relative positions of the internal gear member 18 and the external gear member 15 are preferably maintained. As a result, the efficiency of transmitting the rotational power to the rotary member 12 is improved, and it is preferably possible to prevent foreign matter from entering the region Z during driving.

Next, the operation of the medical rotation mechanism 10 will be described with reference to FIGS. 7 to 10. FIGS. 7 to 10 are cross-sectional views of the medical rotation mechanism 10 for describing an aspect in which the wave generator 14 transmits the rotational power to the rotary member 12.

In the medical rotation mechanism 10, as shown in FIGS. 7 to 10, the external gear member 15 having the external teeth 16 and the internal gear member 18 having the internal teeth 19 function as an external gear and an internal gear that are inscribed and intermeshed at two positions. Since the number of internal teeth 19 is two more than the number of external teeth 16, the medical rotation mechanism 10 functions as a deceleration mechanism. As shown in FIG. 5, the reduction ratio of the medical rotation mechanism 10 in the present embodiment is (20−18)/20=1/10 from (the number of internal teeth 19—the number of external teeth 16)/the number of internal teeth 19.

In FIG. 7, the external tooth 16 and the internal tooth 19 in one of the two inscribed meshing portions E are designated as the external tooth 16 of the number “1” and the internal tooth 19 of the number “1”. In addition, as shown in FIG. 7, each of the external teeth 16 and the internal teeth 19 is assigned a number consecutive from the number “1” along the clockwise direction in the circumferential direction. Here, with respect to the internal tooth 19, a number is assigned to a valley portion that meshes with the external tooth 16.

In FIG. 7, the external tooth 16 of the number “1” and the valley portion of the internal tooth 19 of the number “1” are inscribed and intermeshed with each other. In addition, the valley portion of the external tooth 16 of the number “10” and the internal tooth 19 of the number “11” are also inscribed and intermeshed with each other. In this state, the wave generator 14 is rotated clockwise about the central axis O, and the cam portion 14a is moved clockwise. As a result, the cam portion 14a moves in the circumferential direction, and one of the cam portions 14a pushes the external tooth 16 of the number “2” outward in the radial direction. In addition, the other of the cam portions 14a pushes the external tooth 16 of the number “11” outward in the radial direction.

One of the cam portions 14a moves in the circumferential direction, and the force for pushing the external tooth 16 of the number “1” outward in the radial direction gradually weakens. As a result, the external tooth 16 having the number “1” does not inscribe and mesh with the valley portion of the internal tooth 19 having the number “1”. In addition, the other of the cam portions 14a moves in the circumferential direction, and the force for pushing the external tooth 16 of the number “10” outward in the radial direction gradually weakens. As a result, the external tooth 16 having the number “10” does not inscribe and mesh with the valley portion of the internal tooth 19 having the number “11”.

Next, the external tooth 16 of the number “2” approaches the valley portion of the internal tooth 19 of the number “2”. The internal gear member 18 having an inner circumferential surface formed in a curved shape along the circumferential direction rotates clockwise about the central axis O when the external tooth 16 of the number “2” approaches the valley portion of the internal tooth 19 of the number “2”. As a result, the external tooth 16 of the number “2” and the valley portion of the internal tooth 19 of the number “2” are closer to each other, and are inscribed and intermeshed with each other.

In addition, the external tooth 16 of the number “11” approaches the valley portion of the internal tooth 19 of the number “12”. The internal gear member 18 having an inner circumferential surface formed in a curved shape along the circumferential direction rotates clockwise about the central axis O when the external tooth 16 of the number “11” approaches the valley portion of the internal tooth 19 of the number “12”. As a result, the external tooth 16 of the number “11” and the valley portion of the internal tooth 19 of the number “12” are closer to each other, and are inscribed and intermeshed with each other.

In this manner, by rotating the wave generator 14 about the central axis O, the cam portion 14a moves in the circumferential direction, and the inscribed meshing portion E in which the external tooth 16 and the valley portions of the internal tooth 19 are inscribed and intermeshed moves in the circumferential direction.

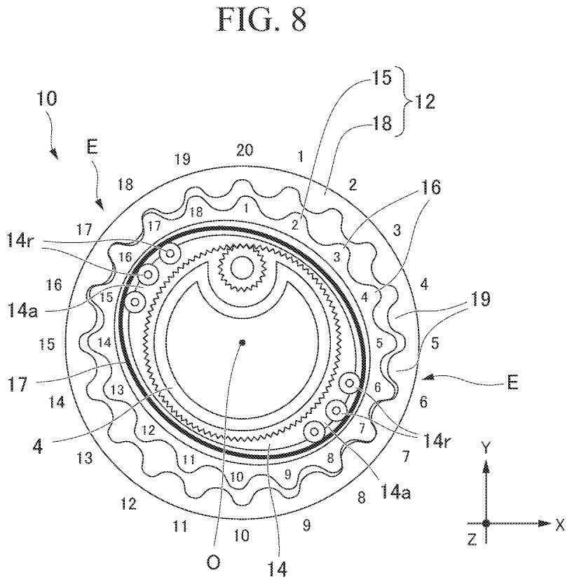

FIG. 8 is a cross-sectional view of the medical rotation mechanism 10 in which the wave generator 14 is further rotated and one of the inscribed meshing portions E is a valley portion of the external tooth 16 of the number “7” and the internal tooth 19 of the number “7”. Compared with the internal gear member 18 shown in FIG. 7, the internal gear member 18 rotates clockwise about the central axis O.

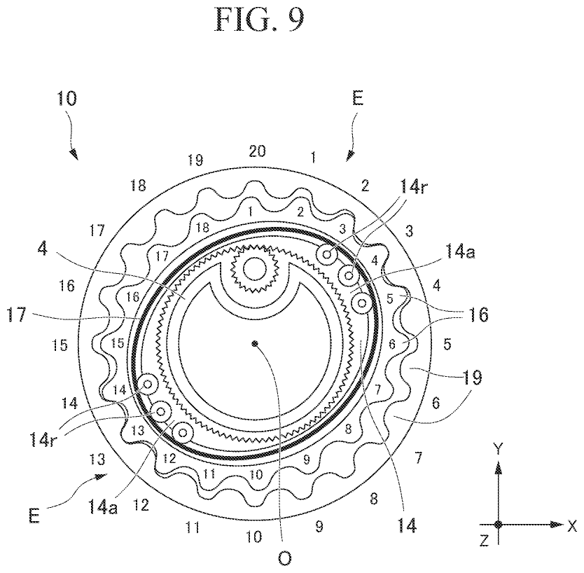

FIG. 9 is a cross-sectional view of the medical rotation mechanism 10 in which the wave generator 14 is further rotated and one of the inscribed meshing portions E is a valley portion of the external tooth 16 of the number “13” and the internal tooth 19 of the number “13”. Compared with the internal gear member 18 shown in FIG. 8, the internal gear member 18 rotates clockwise about the central axis O.

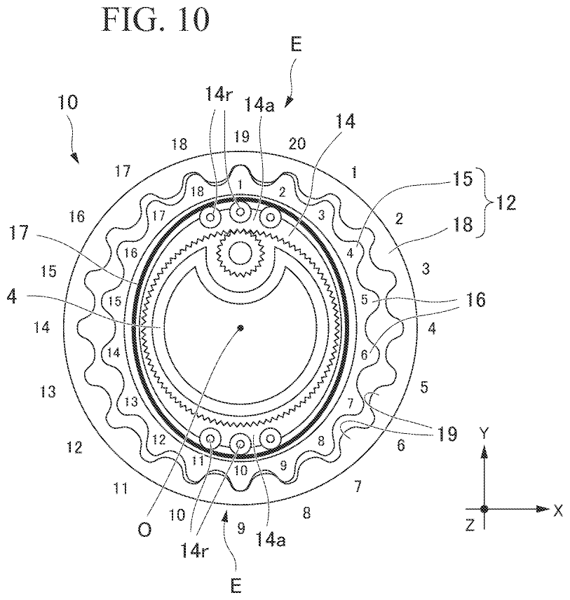

FIG. 10 is a cross-sectional view of the medical rotation mechanism 10 in which the wave generator 14 is rotated 360 degrees. In the medical rotation mechanism 10 in which the wave generator 14 is rotated 360 degrees, one of the inscribed meshing portions E is a valley portion of the external tooth 16 of the number “1” and the internal tooth 19 of the number “19”. Even when the wave generator 14 rotates 360 degrees about the central axis O, the internal gear member 18 does not rotate once. That is, the medical rotation mechanism 10 functions as a deceleration mechanism. When the wave generator 14 rotate once, the internal gear member 18 rotates by two internal teeth 19.

As shown in FIGS. 7 to 10, the rotation center of the wave generator 14 (central axis O) and the rotation center of the internal gear member 18 coincide with each other.

Since the medical rotation mechanism 10 includes the inscribed meshing portions E at two positions facing each other with the central axis O interposed therebetween, the meshing accuracy is high. In addition, the medical rotation mechanism 10 has a large number of meshing teeth and can output a high torque.

According to the endoscope device 100 of the present embodiment, the deceleration mechanism can be provided in the insertion portion 2 after securing the channel 20 having a sufficient space for inserting the treatment tool or the like inside the insertion portion 2. In addition, since the medical rotation mechanism 10 can obtain a large reduction ratio, it is easy to reduce the size and diameter of the medical rotation mechanism 10.

According to the endoscope device 100 of the present embodiment, when the rotary member 12 is removed from the medical rotation mechanism 10, protrusions such as the external teeth 16 and the internal teeth 19 are not exposed to the outside. Therefore, it is preferably possible to prevent foreign matter from being mixed when the rotary member 12 is attached or detached.

According to the endoscope device 100 of the present embodiment, the rotation center of the wave generator 14 and the rotation center of the internal gear member 18 coincide with each other. Therefore, when the operator introduces the insertion portion 2 into the lumen, the spiral tube 9 can be rotated about the rotation axis O, and is easy to handle.

Although the first embodiment of the present invention is described in detail with reference to the drawings, the specific configuration is not limited to the embodiment, and includes design changes and the like within a range that does not deviate from the gist of the present invention. In addition, the components shown in the above-described embodiments and modification examples can be appropriately combined and configured.

### Modification Example 1

In the above embodiment, the wave generator 14 includes the plurality of rollers 14r, and an aspect of the wave generator (first tubular member) is not limited thereto. The wave generator may not include the roller 14r. The wave generator can transmit the rotational power by rotating the cam portion in the circumferential direction.

### Modification Example 2

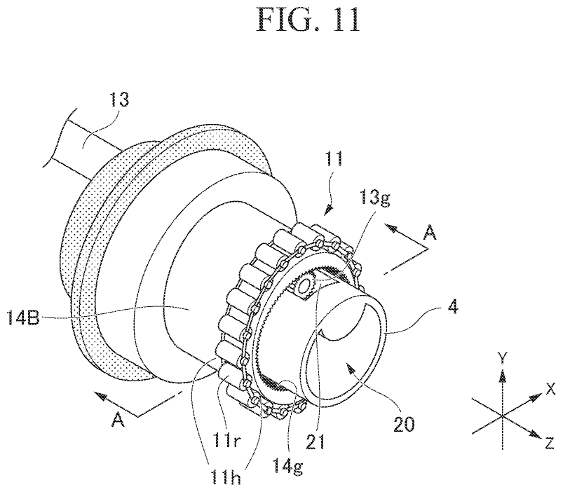

In the above embodiment, the plurality of rollers 14r provided on the wave generator 14 can rotate about the rotation axis of each roller 14r, but cannot rotate with respect to the central axis O of the wave generator 14. An aspect of the endoscope device (medical instrument) is not limited thereto. The endoscope device (medical instrument) may further include the rolling bearing portion 11 shown in FIG. 11. The rolling bearing portion 11 includes a pair of ring-shaped holding portions 11h and a plurality of rollers 11r rotatably held between the pair of holding portions. The roller 11r rotates about a central axis parallel to the central axis O. The plurality of rollers 11r are evenly disposed in the circumferential direction of the pair of holding portions 11h. As shown in FIG. 11, a wave generator 14B, which is a modification example of the wave generator 14, is not provided with the roller 14r, and the rolling bearing portion 11 is fitted on the outer circumference. The rolling bearing portion 11 is not fixed to the wave generator 14B and can rotate about the central axis O with respect to the wave generator 14B. The rolling bearing portion 11 can preferably reduce the friction between the covering member 17 and the wave generator 14B during rotation.

### Modification Example 3

In the above embodiment, the wave generator 14 transmits the rotational power to the rotary member 12 by rotating about the central axis O, and an aspect of the wave generator (first tubular member) is not limited thereto. The wave generator may not rotate. The wave generator may have a configuration in which, for example, the outer circumferential diameter dimension is periodically deformed, and the rotational power may be transmitted by moving a portion corresponding to the cam portion in the circumferential direction.

### Modification Example 4

In the above embodiment, the internal gear member 18 includes the recessed portion 18a and the recessed portion 18b on the inner circumferential surface, and the external gear member 15 includes the projection portion 15a and the projection portion 15b on the outer circumferential surface. An aspect of the recessed portion and the projection portion is not limited thereto. The internal gear member may include a projection portion and the external gear member may include a recessed portion.

### Modification Example 5

In the above embodiment, the wave generator 14 has an elliptical outer circumference in the A-A cross section, and an aspect of the wave generator (first tubular member) is not limited thereto. The wave generator may have, for example, a circular outer circumference in the A-A cross section, and may have rollers at positions facing each other with the central axis O interposed therebetween. The portion provided with the roller functions as a “cam portion” having a length in the radial direction longer than that of the outer circumference having a circular cross section. That is, the wave generator may include a cam portion having a length in the radial direction longer than that of the other portion in the circumferential direction in a part in the circumferential direction.

### Second Embodiment

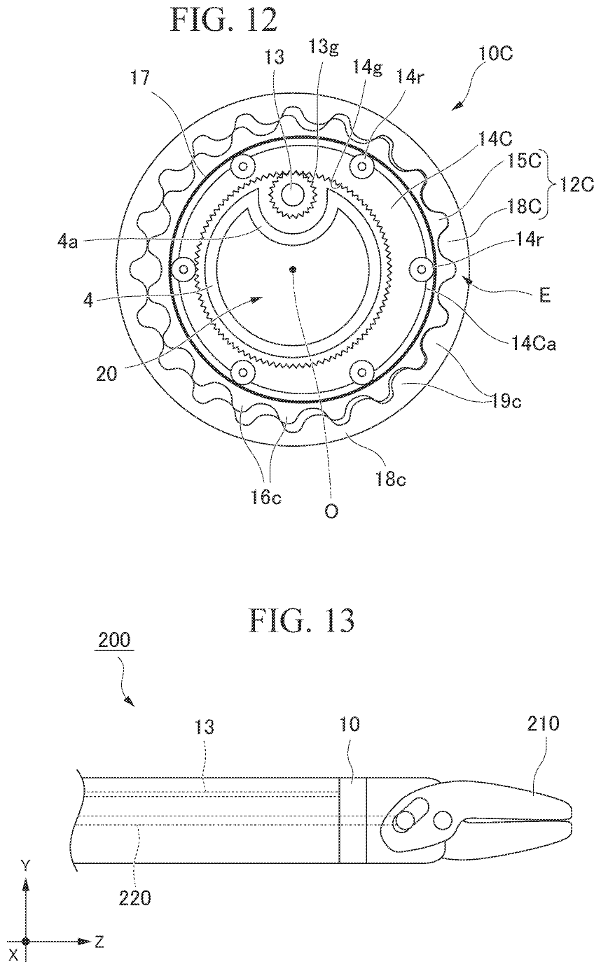

A second embodiment of the present invention will be described with reference to FIG. 12. In the following description, the same reference numerals will be given to the configurations common to those already described, and duplicate descriptions will be omitted. In the present embodiment, an aspect of the wave generator (first tubular member) of the medical rotation mechanism is different from that in the first embodiment.

A medical instrument 100C according to the present embodiment is provided with the insertion portion 2 to be inserted into the lumen of a living body and the operation portion 3 provided on the base end side of the insertion portion 2.

The insertion portion 2C is provided with the long insertion portion main body (main body) 4 extending along the longitudinal axis direction of the insertion portion 2, the curved portion 5 provided on a distal end side of the insertion portion main body 4, the in-vivo insertion mechanism 6, and the medical rotation mechanism 10C.

FIG. 12 is a cross-sectional view of the medical rotation mechanism 10C.

As shown in FIG. 12, the medical rotation mechanism 10C includes the drive gear 13g connected to the shaft 13, a wave generator (first tubular member) 14C inscribed and intermeshed with the drive gear 13g, and the covering member 17 covering the wave generator 14C, and a rotary member (second tubular member) 12C.

Similarly to the wave generator 14, the wave generator 14C is a cylindrical member having transmission gears 14g arranged in the circumferential direction on the inner circumferential surface. The wave generator 14C rotates about the rotation axis O in accordance with the rotation of the drive gear 13g in which the transmission gear 14g is inscribed and intermeshed.

As shown in FIG. 12, the wave generator (first tubular member) 14C includes a cam portion 14Ca having a length in the radial direction longer than that of the other portion in the circumferential direction in a part in the circumferential direction. When the wave generator 14C rotates about the central axis O, the cam portion 14Ca moves in the circumferential direction. The wave generator 14C of the second embodiment includes only one cam portion 14Ca in the circumferential direction.

In addition, the wave generator 14C includes the plurality of rollers 14r. The roller 14r is rotatably supported in the circumferential direction. The plurality of rollers 14r are evenly disposed in the circumferential direction. The wave generator 14C brings the cam portion 14Ca into contact with the rotary member 12C disposed outside the wave generator 14C via the roller 14r, and transmits rotational power about the central axis O of the wave generator 14C to the rotary member 12C.

The rotary member 12C is a cylindrical member that can be attached to or detached from the wave generator 14C, and includes an external gear member 15C and an internal gear member 18C disposed on the outside of the external gear member 15C in the radial direction.

As shown in FIG. 12, the external gear member 15C is a tubular member disposed on the outside of the wave generator 14C in the radial direction and made of metal, reinforced resin, or the like. The external gear member 15C is non-rotatably supported with respect to the insertion portion main body 4. The external gear member 15 swings according to the power of the wave generator 14.

The external gear member 15C includes a plurality of external teeth 16C arranged in the circumferential direction on the outer circumferential surface. On the outer circumferential surface of the external gear member 15C, the external teeth 16C are evenly disposed in the circumferential direction, and the outer circumferential surface including the external teeth 16C forms a cycloid curve or a cycloid parallel curve along the circumferential direction. The number of external teeth 16C is 19.

As shown in FIG. 12, the internal gear member 18C is a tubular member disposed on the outer side of the external gear member 15C in the radial direction, and is made of a highly rigid metal or the like. The internal gear member 18C is rotatably supported with respect to the insertion portion main body 4 about the rotation axis O. The internal gear member 18C is connected to the spiral tube 9, and when the internal gear member 18C rotates about the rotation axis O, the spiral tube 9 also rotates about the rotation axis O.

The internal gear member 18C includes a plurality of internal teeth 19C arranged in the circumferential direction on the inner circumferential surface. As shown in FIG. 12, on the inner circumferential surface of the internal gear member 18C, the internal teeth 19C are evenly disposed in the circumferential direction, and the inner circumferential surface including the internal teeth 19C forms a cycloid curve or a cycloid parallel curve along the circumferential direction. As shown in FIG. 12, the number of internal teeth 19 included in the internal gear member 18 is 20. On the other hand, the number of external teeth 16 included in the external gear member 15 is 19. That is, the number of internal teeth 19 is larger than the number of external teeth 16. The external gear member 15C and the internal gear member 18C function as an “inscribed planetary gear mechanism”.

Due to the contact of the roller 14r near the cam portion 14Ca of the wave generator 14C, the external teeth 16C of the external gear member 15C having the farthest distance from the central axis O of the wave generator 14C are inscribed and intermeshed with the valley of the internal teeth 19C. The wave generator 14C causes the valleys of the external teeth 16C and the internal teeth 19C to be inscribed and intermeshed, and the inscribed meshing portion E in which the external teeth 16C and the internal teeth 19C are inscribed and intermeshed to move in the circumferential direction, and transmits the rotational power about the central axis O of the wave generator 14C to the rotary member 12. As a result, the rotary member 12 rotates about the central axis O.

According to the medical instrument 100C of the present embodiment, similarly to the first embodiment, the deceleration mechanism can be provided in the insertion portion 2 after securing the channel 20 having a sufficient space for inserting the treatment tool or the like inside the insertion portion 2.

According to the medical instrument 100C of the present embodiment, when the rotary member 12 is removed from the medical rotation mechanism 10C, protrusions such as the external teeth 16 and the internal teeth 19 are not exposed to the outside. Therefore, it is preferably possible to prevent foreign matter from being mixed when the rotary member 12 is attached or detached.

Although the second embodiment of the present invention is described in detail with reference to the drawings, the specific configuration is not limited to the embodiment, and includes design changes and the like within a range that does not deviate from the gist of the present invention. In addition, the components shown in the above-described embodiments and modification examples can be appropriately combined and configured together.

### Third Embodiment

A third embodiment of the present invention will be described with reference to FIG. 13. In the following description, the same reference numerals will be given to the configurations common to those already described, and duplicate descriptions will be omitted. The present embodiment is different in that the medical rotation mechanism is provided not in the endoscope device but in a treatment tool.

FIG. 13 is a side view of a treatment tool 200 according to the present embodiment.

The treatment tool (medical instrument) 200 is provided with a pair of forceps 210, an opening and closing operation wire 220, and the medical rotation mechanism 10.

In the treatment tool 200, the shaft 13 rotates and the wave generator 14 rotates about the central axis O, similarly to the endoscope device 100 of the first embodiment. The wave generator 14 rotates the rotary member 12 disposed outside the wave generator 14.

The rotary member 12 is connected to the pair of forceps 210, and when the rotary member 12 rotates about the central axis O, the pair of forceps 210 also rotates about the central axis O.

According to the treatment tool 200 of the present embodiment, the treatment tool 200 having a small diameter dimension can be provided with a medical rotation mechanism 10 having a deceleration mechanism.

According to the treatment tool 200 of the present embodiment, when the rotary member 12 is removed from the medical rotation mechanism 10, protrusions such as the external teeth 16 and the internal teeth 19 are not exposed to the outside. Therefore, it is preferably possible to prevent foreign matter from being mixed therewith.

### Fourth Embodiment

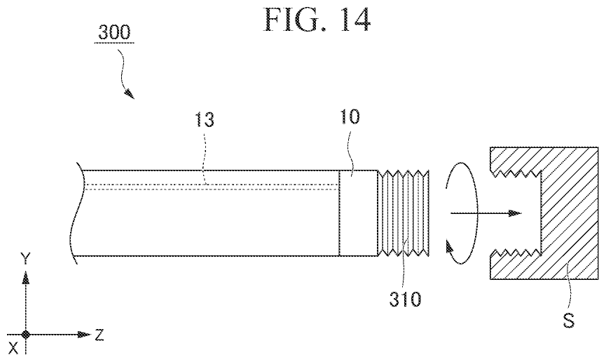

A fourth embodiment of the present invention will be described with reference to FIG. 14. In the following description, the same reference numerals will be given to the configurations common to those already described, and duplicate descriptions will be omitted. The present embodiment is different in that the medical rotation mechanism is provided not in the endoscope device but in a treatment tool.

FIG. 14 is a side view of a treatment tool 300 according to the present embodiment.

The treatment tool (medical instrument) 300 is provided with a feed screw (linear motion mechanism) 310 and the medical rotation mechanism 10.

In the treatment tool 300, the shaft 13 rotates and the wave generator 14 rotates about the central axis O, similarly to the endoscope device 100 of the first embodiment. The wave generator 14 rotates the rotary member 12 disposed outside the wave generator 14.

The rotary member 12 is connected to the feed screw 310, and when the rotary member 12 rotates about the central axis O, the feed screw 310 also rotates about the central axis O. The treatment tool 300 can screw the feed screw 310 into a screw S.

According to the treatment tool 300 of the present embodiment, the medical treatment tool 300 having a small diameter dimension can be provided with a medical rotation mechanism 10 having a deceleration mechanism.

According to the treatment tool 300 of the present embodiment, a rotational motion of the medical rotation mechanism 10 can be converted into the linear motion of the feed screw 310.

According to the treatment tool 300 of the present embodiment, when the rotary member 12 is removed from the medical rotation mechanism 10, protrusions such as the external teeth 16 and the internal teeth 19 are not exposed to the outside. Therefore, it is preferably possible to prevent foreign matter from being mixed therewith.

What is claimed is:

1. A medical instrument comprising:
a main body;
a first tubular member provided on the main body, the first tubular member configured to transmit power by rotating or periodically deforming; and
a second tubular member configured to be attached to or detached from the first tubular member, wherein the second tubular member includes:
an external gear member disposed on an outside of the first tubular member in a radial direction, and configured to have a plurality of external teeth arranged in a circumferential direction on an outer circumferential surface and to swing or deform according to power of the first tubular member; and
an internal gear member disposed on an outside of the external gear member in the radial direction and configured to have a plurality of internal teeth arranged in the circumferential direction on an inner circumferential surface,
wherein the number of internal teeth is greater than the number of external teeth.

2. The medical instrument according to claim 1, further comprising:
an elastic covering member configured to cover the first tubular member between the first tubular member and the second tubular member.

3. The medical instrument according to claim 1, wherein
the second tubular member includes a fin spirally wound around an outer circumference thereof.

4. The medical instrument according to claim 1, wherein
the first tubular member includes a cam having a length in the radial direction longer than that of the other portion in the circumferential direction in a part in the circumferential direction, and
wherein the cam is configured to cause the external teeth and the internal teeth to be inscribed and intermeshed with each other at least one position.

5. The medical instrument according to claim 4, wherein
the first tubular member has an elliptical shape in a cross section which is perpendicular to a longitudinal direction.

6. The medical instrument according to claim 4, wherein
the external gear member is deformed into the elliptical shape according to the power of the first tubular member.

7. The medical instrument according to claim 1, wherein
the first tubular member includes a roller on an outer circumference.

8. The medical instrument according to claim 1, further comprising:
a rolling bearing portion configured to fit on an outer circumference of the first tubular member.

9. The medical instrument according to claim 1, wherein
the external gear member includes a projection portion formed in an annular shape on the outer circumferential surface, and
the internal gear member includes a recessed portion formed in an annular shape on the inner circumferential surface and configured to engage with the projection portion.

10. The medical instrument according to claim 1, wherein
the external gear member includes a recessed portion formed in an annular shape on the outer circumferential surface, and
the internal gear member includes a projection portion formed in an annular shape on the inner circumferential surface and configured to engage with the recessed portion.

11. The medical instrument according to claim 9, further comprising:
a base end side engaging portion in which the projection portion and the recessed portion engage with each other on a base end side; and
a distal end side engaging portion in which the projection portion and the recessed portion engage with each other on a distal end side, wherein
the internal tooth and the external tooth mesh with each other in a region between the base end side engaging portion and the distal end side engaging portion.

12. The medical instrument according to claim 1, wherein
the outer circumferential surface of the external gear member and the inner circumferential surface of the internal gear member form an involute curve along the circumferential direction.

13. The medical instrument according to claim 1, wherein
the outer circumferential surface of the external gear member and the inner circumferential surface of the internal gear member form a cycloid curve or a cycloid parallel curve along the circumferential direction.

14. The medical instrument according to claim 1, further comprising:
a linear motion mechanism configured to convert a rotational motion of the second tubular member into a linear motion.

15. The medical instrument according to claim 14, wherein
the linear motion mechanism is a feed screw.
